(19) United States
(12) Patent Application Publication
(10) Pub. No.: US 2024/0325576 A1
(43) Pub. Date: Oct. 3, 2024

(54) STERILIZER SYSTEM FOR GROOMING IMPLEMENTS

(71) Applicants: Cristopher Martinez, Bronx, NY (US); Annerys Heyer-Jaquez, Bronx, NY (US)

(72) Inventors: Cristopher Martinez, Bronx, NY (US); Annerys Heyer-Jaquez, Bronx, NY (US)

(21) Appl. No.: 18/127,090

(22) Filed: Mar. 28, 2023

(51) Int. Cl. A61L 2/10

(52) U.S. Cl. CPC A61L 2/10; A61L 2202/11; A61L 2202/121; A61L 2202/122

(57) ABSTRACT

A sterilizer system for sterilizing grooming implements with ultraviolet light includes a housing including a base wall and a perimeter wall. The perimeter wall is coupled to and extends away from the base wall and bounds a receiving space. A light emitting assembly is mounted on the housing and is configured to emit sterilizing light into the receiving space. A container is removably positioned in the receiving space and comprises a bottom wall and a peripheral wall. The peripheral wall is coupled to and extends away from the bottom wall and bounds a containment space. A plurality of grooming implements is removably positioned in the containment space.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sterilizer systems and more particularly pertains to a new sterilizer system for sterilizing grooming implements with ultraviolet light.

### (2) Description of Related Art Including Information Disclosed Under 37 CFR 1.97 and 1.98

The prior art discloses several examples of sterilizer systems for sterilizing grooming implements with ultraviolet light. These systems typically include a housing with a receiving space for positioning grooming implements inside and light emitters directed to emit ultraviolet light into the receiving space. The prior art describes such a system with a lid that is pivotally attached to the housing to cover the receiving space. A light emitter is positioned on the lid of this system such that the grooming implements are exposed to sanitizing light from above the grooming implements. However, the prior art dose not disclose such a device with a pivoting lid that surrounds the receiving space in a plurality of light emitters and which provides a container which holds the grooming implements and is removably positionable in the receiving space. The prior art also fails to disclose a sterilizer system which includes indicators that display operational information of the sterilizer system.

### BRIEF SUMMARY OF THE INVENTION

An embodiment of the disclosure meets the needs presented above by generally comprising a housing including a base wall and a perimeter wall. The perimeter wall is coupled to and extends away from the base wall and bounds a receiving space. A light emitting assembly is mounted on the housing and is configured to emit sterilizing light into the receiving space. A container is removably positioned in the receiving space and comprises a bottom wall and a peripheral wall. The peripheral wall is coupled to and extends away from the bottom wall and bounds a containment space. A plurality of grooming implements is removably positioned in the containment space.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a sterilizer system according to an embodiment of the disclosure.

FIG. 2 is a perspective view of an embodiment of the disclosure.

FIG. 3 is an exploded perspective view of an embodiment of the disclosure.

FIG. 4 is a top view of an embodiment of the disclosure.

FIG. 5 is a block diagram of an embodiment of the disclosure.

FIG. 6 is a perspective view of an embodiment of the disclosure.

### DETAILED DESCRIPTION OF THE INVENTION

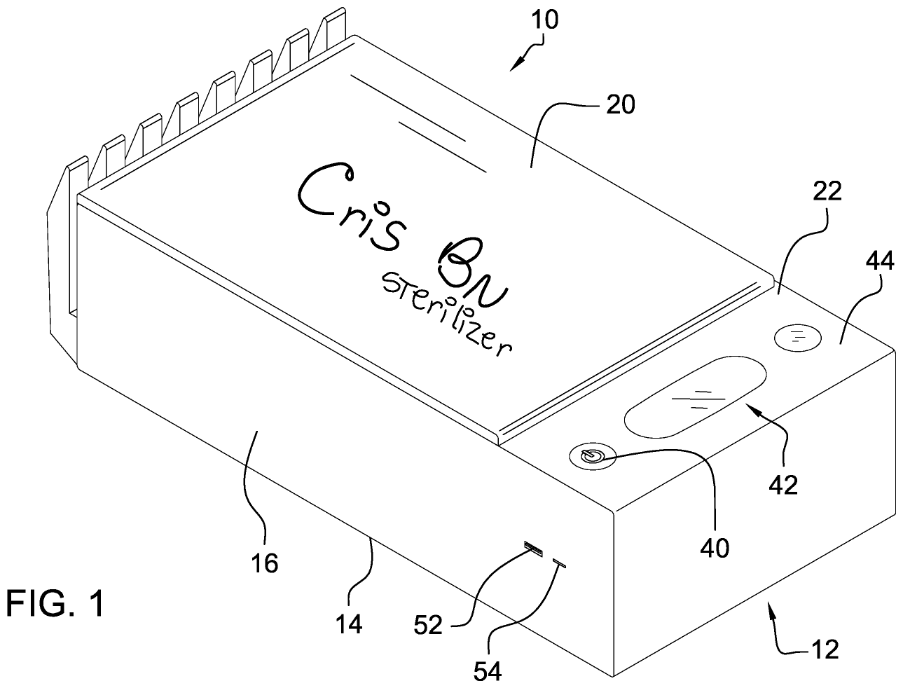

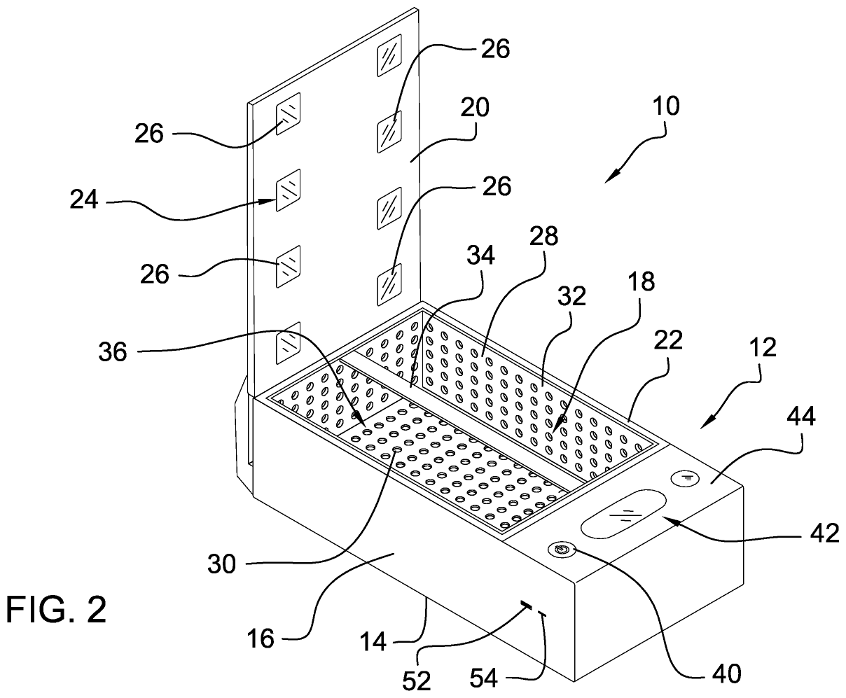

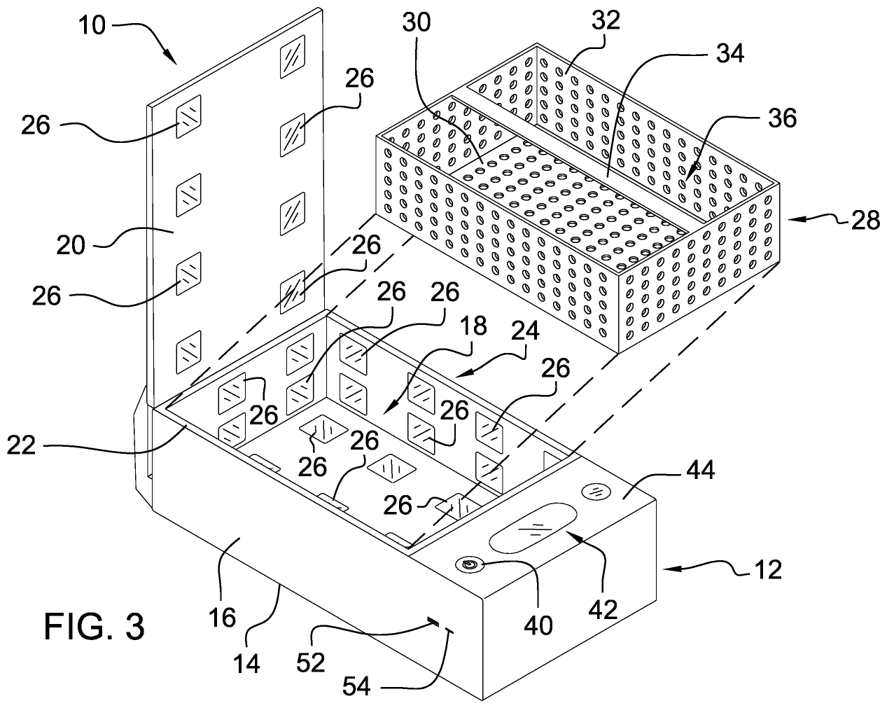

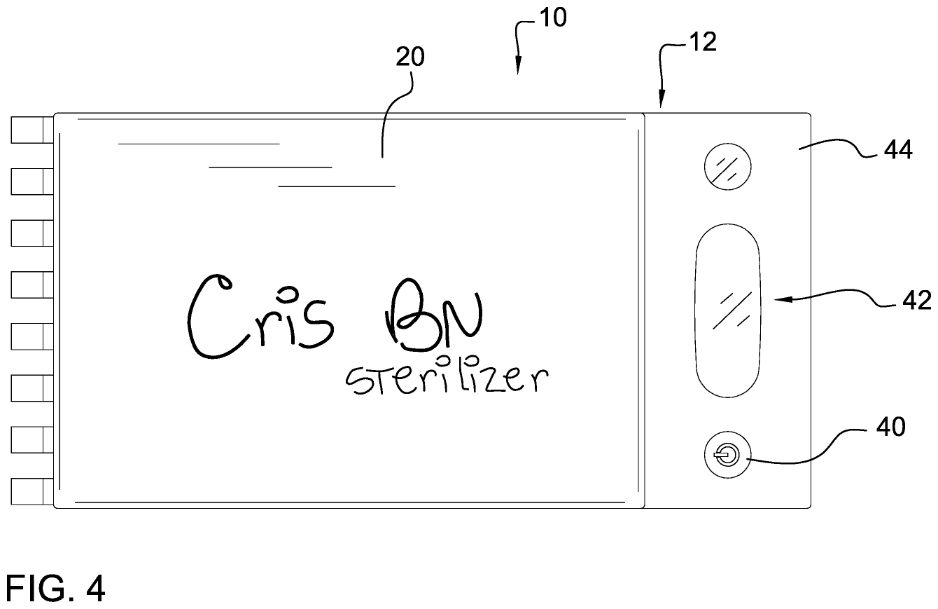

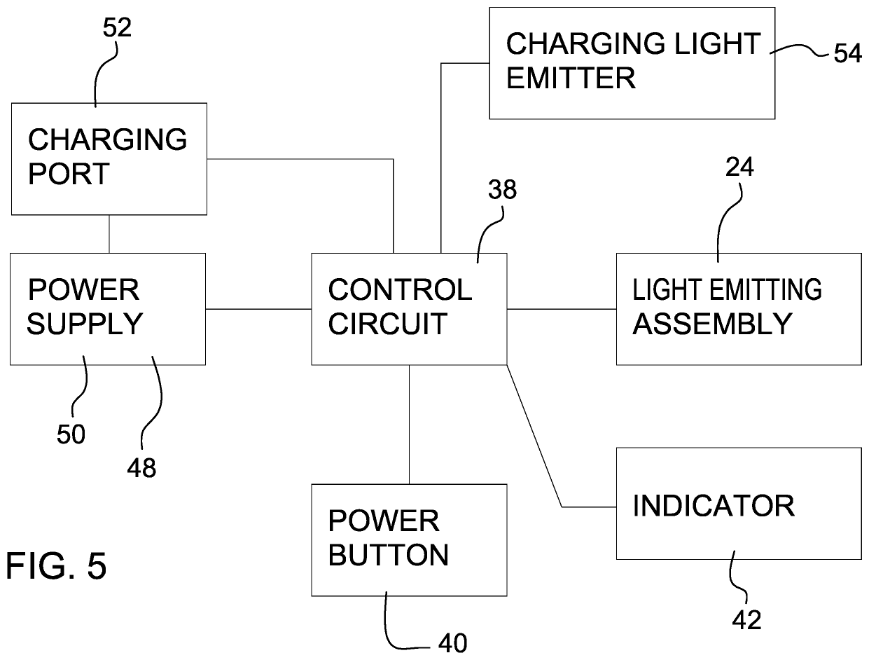

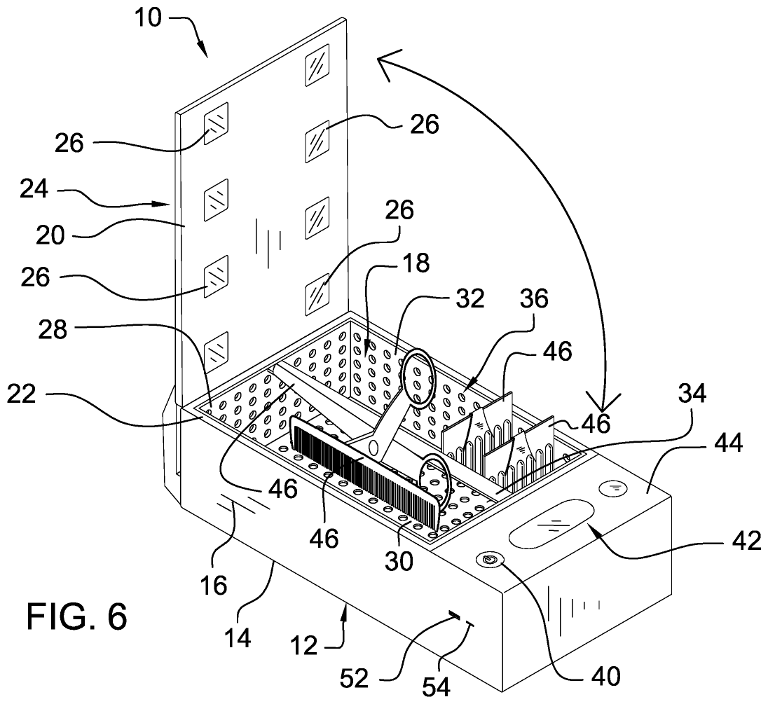

With reference now to the drawings, and in particular to FIGS. 1 through 6 thereof, a new sterilizer system embodying the principles and concepts of an embodiment of the disclosure and generally designated by the reference numeral 10 will be described.

As best illustrated in FIGS. 1 through 6, the sterilizer system 10 generally comprises a housing 12 including a base wall 14 and a perimeter wall 16 coupled to and extending away from the base wall 14. The perimeter wall 16 bounds a receiving space 18, and the housing 12 also includes a lid 20 which is positioned on an upper edge 22 of the perimeter wall 16 and is movable to alternately open and close the receiving space 18. The lid 20 is pivotally coupled to the housing 12.

A light emitting assembly 24 is mounted on the housing 12 and is configured to emit sterilizing light into the receiving space 18. The light emitting assembly 24 comprises a plurality of light emitters 26, each of which is configured to emit ultraviolet light. Each of the base wall 14, the perimeter wall 16, and the lid 20 has at least one light emitter 26 of the plurality of light emitters 26 mounted thereto, and the plurality of light emitters 26 is arranged to surround the receiving space 18.

A container 28 is removably positioned in the receiving space 18. The container 28 comprises a bottom wall 30, a peripheral wall 32, and a handle 34. The peripheral wall 32 is coupled to and extends away from the bottom wall 30 and bounds a containment space 36. The handle 34 is coupled to the peripheral wall 32, extends across the containment space 36, and is spaced from the bottom wall 30.

A control circuit 38 is electrically coupled to the light emitting assembly 24, and a power button 40 is electrically coupled to the control circuit 38 and positioned on an exterior 44 of the housing 12. The power button 40 is actuatable to activate the light emitting assembly 24 for a predetermined period of time. The predetermined period of time may be selectable by the user via an input or may be determined during manufacture. The light emitting assembly 24 also may be actuatable to activate the light emitting assembly 24 indefinitely.

An indicator 42 is electrically coupled to the control circuit 38. The indicator 42 displays operation information which may include an indication of when the light emitting assembly 24 is activated, an amount of time remaining before the light emitting assembly 24 will be deactivated, an amount of time the light emitting assembly 24 continuously emitted light, or the like. The indicator 42 may display amounts of time with a numerical representation, a graphical representation of the proportion of time relative to the predetermined period of time, or the like. The indicator 42 comprises a plurality of indication lights positioned on the exterior 44 of the housing 12. In some embodiments, the indication lights may be arranged along a line and consecutively deactivate over the course of the predetermined period of time to indicate the proportion of time which has elapsed since activating the light emitting assembly 24 relative to the predetermined period of time.

A plurality of grooming implements 46 is removably positioned in the containment space 36. A power supply 48 is electrically coupled to the control circuit 38 and comprises a battery 50 which is positioned in the housing 12. The power supply 48 may be external to the housing 12 and may be decouplable from the control circuit 38. A charging port 52 is electrically coupled to the control circuit 38 and is positioned on the exterior 44 of the housing 12 such that the battery 50 may be charged by an external power source. A charging light emitter 54 is positioned on the exterior 44 of the housing 12 adjacent to the charging port 52 and configured to indicate when the charging port 52 is coupled to the external power source.

In use, the plurality of grooming implements 46 are positioned in the containment space 36 of the container 28, and the container 28 is positioned in the receiving space 18 of the housing 12. The lid 20 of the housing 12 is shut, and the power button 40 is actuated to activate the light emitting assembly 24 for the predetermined period of time. The grooming implements 46 are removed from the containment space 36 after the predetermined period of time has elapsed.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sterilizer system comprising:
a housing including a base wall and a perimeter wall being coupled to and extending away from the base wall, the perimeter wall bounding a receiving space;
a light emitting assembly being mounted on the housing and being configured to emit sterilizing light into the receiving space;
a container being removably positioned in the receiving space, the container comprising a bottom wall and a peripheral wall, the peripheral wall being coupled to and extending away from the bottom wall, the peripheral wall bounding a containment space; and
a plurality of grooming implements being removably positioned in the containment space.

2. The system of claim 1, wherein the housing further comprises a lid being positioned on an upper edge of the perimeter wall and being movable to alternately open and close the receiving space.

3. The system of claim 2, wherein the lid is pivotally coupled to the housing.

4. The system of claim 1, wherein the light emitting assembly comprises a plurality of light emitters, each light emitter being configured to emit ultraviolet light.

5. The system of claim 4, wherein the housing further comprises a lid being positioned on an upper edge of the perimeter wall and being movable to alternately open and close the receiving space, each of the base wall, the perimeter wall, and the lid having at least one light emitter of the plurality of light emitters mounted thereto.

6. The system of claim 5, wherein the plurality of light emitters is arranged to surround the receiving space.

7. The system of claim 1, wherein the container further comprises a handle being coupled to the peripheral wall and extending across the containment space, the handle being spaced from the bottom wall.

8. The system of claim 1, further comprising:
a control circuit being electrically coupled to the light emitting assembly; and
a power button being electrically coupled to the control circuit, the power button being actuatable to activate the light emitting assembly for a predetermined period of time.

9. The system of claim 8, further comprising an indicator being electrically coupled to the control circuit, the indicator displaying operation information.

10. The system of claim 9, wherein the indicator comprises a plurality of indication lights.

11. The system of claim 10, wherein the power button and the plurality of indication lights are positioned on an exterior of the housing.

12. The system of claim 9, further comprising a power supply being electrically coupled to the control circuit.

13. The system of claim 12, wherein the power supply comprises a battery.

14. The system of claim 13, further comprising a charging port being electrically coupled to the control circuit and being positioned on the exterior of the housing, the battery being positioned in the housing.

15. A sterilizer system comprising:
a housing including:
a base wall;
a perimeter wall being coupled to and extending away from the base wall, the perimeter wall bounding a receiving space; and
a lid being positioned on an upper edge of the perimeter wall and being movable to alternately open and close the receiving space, the lid being pivotally coupled to the housing;
a light emitting assembly being mounted on the housing and being configured to emit sterilizing light into the receiving space, the light emitting assembly comprising a plurality of light emitters, each light emitter being configured to emit ultraviolet light, each of the base wall, the perimeter wall, and the lid having at least one light emitter of the plurality of light emitters mounted thereto, the plurality of light emitters being arranged to surround the receiving space;
a container being removably positioned in the receiving space, the container comprising a bottom wall, a peripheral wall, and a handle, the peripheral wall being coupled to and extending away from the bottom wall, the peripheral wall bounding a containment space, the handle being coupled to the peripheral wall and extending across the containment space, the handle being spaced from the bottom wall;
a control circuit being electrically coupled to the light emitting assembly;
a power button being electrically coupled to the control circuit, the power button being actuatable to activate the light emitting assembly for a predetermined period of time, the power button being positioned on an exterior of the housing;
an indicator being electrically coupled to the control circuit, the indicator displaying operation information, the indicator comprising a plurality of indication lights positioned on the exterior of the housing;
a plurality of grooming implements being removably positioned in the containment space;
a power supply being electrically coupled to the control circuit, the power supply comprising a battery positioned in the housing; and
a charging port being electrically coupled to the control circuit and being positioned on the exterior of the housing.
